(19) United States
(12) Patent Application Publication
(10) Pub. No.: US 2006/0258597 A1
(43) Pub. Date: Nov. 16, 2006

(54) Antitumorigenic drug combination and dosing schedule

(76) Inventors: Jack Henkin, Highland Park, IL (US); Abdullah W. Kherzai, Lake Villa, IL (US); Laura M. McKay, Lake Villa, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/393,466

(22) Filed: Mar. 30, 2006

Related U.S. Application Data
(60) Provisional application No. 60/666,492, filed on Mar. 30, 2005.

(51) Int. Cl. A61K 38/08

(52) U.S. Cl. 514/16

(57) ABSTRACT

Compositions comprising drugs having additive antitumorigenesis activity and methods of treatment using the combinations is disclosed.

This application claims priority from U.S. Provisional Patent Application Ser. No. 60/666,492, filed on Mar. 30, 2005, incorporated herein by reference.

### FIELD OF THE INVENTION

The invention relates to compositions comprising drugs having additive antitumorigenesis activity and methods of treatment using the combinations.

### BACKGROUND OF THE INVENTION

Physiologic angiogenesis is associated with normal events such as reproduction and wound repair. Pathologic angiogenesis, however, may cause or exacerbate diseases such as cancer. There is therefore an existing need in the therapeutic arts for treatment of diseases which are caused or exacerbated by pathologic angiogenesis.

### BRIEF DESCRIPTION OF THE FIGURE

FIG. 1 shows comparive antitumorigenesis of bevacizumab and N-Ac-Sar-Gly-Val-D-alloIle-Thr-Nva-Ile-Arg-Pro-NHCH₂CH₃ compared to bevacizumab or N-Ac-Sar-Gly-Val-D-alloIle-Thr-Nva-Ile-Arg-Pro-NHCH₂CH₃ in the HT29-Colon Model.

### SUMMARY OF THE INVENTION

Accordingly, one embodiment of this invention pertains to compositions for treating cancer in a mammal with measurably additive antitumorigenesis, said compositions comprising therapeutically effective amounts of a vascular endothelial growth factor (VEGF) inhibitor and a peptidomimetic of the second of the three Type-1 repeats of thrombospondin-1 (TSP-1) or a salt, prodrug or salt of a prodrug of either or both.

Still another embodiment pertains to separate compositions to be administered together for treating cancer in a mammal with measurably additive antitumorigenesis, one comprising a therapeutically effective amount of a VEGF inhibitor or a salt, prodrug, or salt of a prodrug thereof, and the other comprising a peptidomimetic of the second of the three Type-1 repeats of TSP-1 or a salt, prodrug or salt of a prodrug thereof.

Still another embodiment pertains to compositions for treating cancer in a mammal with measurably additive antitumorigenesis, said compositions comprising therapeutically effective amounts of a VEGF inhibitor and Sar-Gly-Val-D-alloIle-Thr-Nva-Ile-Arg-Pro or a salt, prodrug or salt of a prodrug of either or both.

Still another embodiment pertains to separate compositions to be administered together for treating cancer in a mammal with measurably additive antitumorigenesis, one comprising a therapeutically effective amount of a VEGF inhibitor or a salt, prodrug or salt of a prodrug thereof, and the other comprising Sar-Gly-Val-D-alloIle-Thr-Nva-Ile-Arg-Pro or a salt, prodrug or salt of a prodrug thereof.

Still another embodiment pertains to compositions for treating cancer in a mammal with measurably additive antitumorigenesis, said compositions comprising therapeutically effective amounts of bevacizumab (AVASTIN®) and a peptidomimetic of the second of the three Type-1 repeats of TSP-1 or a salt, prodrug, or salt of a prodrug thereof.

Still another embodiment pertains to separate compositions to be administered together for treating cancer in a mammal with measurably additive antitumorigenesis, one comprising a therapeutically effective amount of bevacizumab and the other comprising a peptidomimetic of the second of the three Type-1 repeats of TSP-1 or a salt, prodrug, or salt of a prodrug thereof.

Still another embodiment pertains to compositions for treating cancer in a mammal with measurably additive antitumorigenesis, said compositions comprising therapeutically effective amounts of bevacizumab and Sar-Gly-Val-D-alloIle-Thr-Nva-Ile-Arg-Pro or a salt, prodrug, or salt of a prodrug thereof.

Still another embodiment pertains to separate compositions to be administered together for treating cancer in a mammal with measurably additive antitumorigenesis, one comprising a therapeutically effective amount of bevacizumab and the other comprising Sar-Gly-Val-D-alloIle-Thr-Nva-Ile-Arg-Pro or a salt, prodrug, or salt of a prodrug thereof.

Still another embodiment pertains to compositions for inhibiting tumor growth in a mammal with measurably additive antitumorigenesis, said compositions comprising therapeutically effective amounts of a VEGF inhibitor and a peptidomimetic of the second of the three Type-1 repeats of TSP-1 or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of a VEGF inhibitor or a salt, prodrug, or salt of a prodrug thereof, and the other comprising a peptidomimetic of the second of the three Type-1 repeats of TSP-1 or a salt, prodrug or salt of a prodrug thereof.

Still another embodiment pertains to compositions for inhibiting tumor growth in a mammal with measurably additive antitumorigenesis, said compositions comprising therapeutically effective amounts of a VEGF inhibitor or a salt, prodrug, or salt of a prodrug thereof and Sar-Gly-Val-D-alloIle-Thr-Nva-Ile-Arg-Pro, or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of a VEGF inhibitor or a salt, prodrug or salt of a prodrug thereof, and the other comprising Sar-Gly-Val-D-alloIle-Thr-Nva-Ile-Arg-Pro or a salt, prodrug or salt of a prodrug thereof.

Still another embodiment pertains to compositions for inhibiting tumor growth in a mammal with measurably additive antitumorigenesis, said compositions comprising therapeutically effective amounts of bevacizumab and a peptidomimetic of the second of the three Type-1 repeats of TSP-1, or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of bevacizumab and the other comprising a peptidomimetic of the second of the three Type-1 repeats of TSP-1 or a salt, prodrug, or salt of a prodrug thereof.

Still another embodiment pertains to compositions for inhibiting tumor growth in a mammal with measurably additive antitumorigenesis, said compositions comprising therapeutically effective amounts of bevacizumab and Sar-Gly-Val-D-alloIle-Thr-Nva-Ile-Arg-Pro or a salt, prodrug, or salt of a prodrug thereof.

Still another embodiment pertains to separate compositions to be administered together for inhibiting tumor growth in a mammal with measurably additive antitumorigenesis, one comprising a therapeutically effective amount of bevacizumab and the other comprising Sar-Gly-Val-D-alloIle-Thr-Nva-Ile-Arg-Pro or a salt, prodrug, or salt of a prodrug thereof.

Still another embodiment pertains to compositions for treating fibrosarcoma in a mammal with measurably additive antitumorigenesis, said compositions comprising therapeutically effective amounts of a VEGF inhibitor and a peptidomimetic of the second of the three Type-1 repeats of TSP-1 or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of a VEGF inhibitor or a salt, prodrug, or salt of a prodrug thereof, and the other comprising a peptidomimetic of the second of the three Type-1 repeats TSP-1 or a salt, prodrug or salt of a prodrug thereof.

Still another embodiment pertains to compositions for treating fibrosarcoma in a mammal with measurably additive antitumorigenesis, said compositions comprising therapeutically effective amounts of a VEGF inhibitor and Sar-Gly-Val-D-alloIle-Thr-Nva-Ile-Arg-Pro or a salt, prodrug, or salt of a prodrug of either or both.

Still another embodiment pertains to separate compositions to be administered together for treating fibro sarcoma in a mammal with measurably additive antitumorigenesis, one comprising a therapeutically effective amount of a VEGF inhibitor or a salt, prodrug or salt of a prodrug thereof, and the other comprising Sar-Gly-Val-D-alloIle-Thr-Nva-Ile-Arg-Pro or a salt, prodrug or salt of a prodrug thereof.

Still another embodiment pertains to compositions for treating fibrosarcoma in a mammal with measurably additive antitumorigenesis, said compositions comprising therapeutically effective amounts of bevacizumab and a peptidomimetic of the second of the three Type-1 repeats of TSP-1 or a salt, prodrug, or salt of a prodrug thereof.

Still another embodiment pertains to separate compositions to be administered together for treating fibro sarcoma in a mammal with measurably additive antitumorigenesis, one comprising a therapeutically effective amount of bevacizumab and the other comprising a peptidomimetic of the second of the three Type-1 repeats of TSP-1 or a salt, prodrug, or salt of a prodrug thereof.

Still another embodiment pertains to compositions for treating fibrosarcoma in a mammal with measurably additive antitumorigenesis, said compositions comprising therapeutically effective amounts of bevacizumab and Sar-Gly-Val-D-alloIle-Thr-Nva-Ile-Arg-Pro, or a salt, prodrug, or salt of a prodrug thereof.

Still another embodiment pertains to separate compositions to be administered together for treating fibrosarcoma in a mammal with measurably additive antitumorigenesis, one comprising a therapeutically effective amount of bevacizumab and the other comprising Sar-Gly-Val-D-alloIle-Thr-Nva-Ile-Arg-Pro or a salt, prodrug, or salt of a prodrug thereof.

Still another embodiment pertains to compositions for treating colon cancer in a mammal with measurably additive antitumorigenesis, said compositions comprising therapeutically effective amounts of a VEGF inhibitor and a peptidomimetic of the second of the three Type-1 repeats of TSP-1 or a salt, prodrug, or salt of a prodrug of either or both.

Still another embodiment pertains to separate compositions to be administered together for treating colon cancer in a mammal with measurably additive antitumorigenesis, one comprising a therapeutically effective amount of a VEGF inhibitor or a salt, prodrug, or salt of a prodrug thereof, and the other comprising a peptidomimetic of the second of the three Type-1 repeats of TSP-1 or a salt, prodrug or salt of a prodrug thereof.

Still another embodiment pertains to compositions for treating colon cancer in a mammal with measurably additive antitumorigenesis, said compositions comprising therapeutically effective amounts of a VEGF inhibitor and Sar-Gly-Val-D-alloIle-Thr-Nva-Ile-Arg-Pro or a salt, prodrug, or salt of a prodrug of either or both.

Still another embodiment pertains to separate compositions to be administered together for inhibiting tumor growth in a mammal with measurably additive antitumorigenesis, one comprising a therapeutically effective amount of a VEGF inhibitor or a salt, prodrug or salt of a prodrug thereof, and the other comprising Sar-Gly-Val-D-alloIle-Thr-Nva-Ile-Arg-Pro or a salt, prodrug or salt of a prodrug thereof.

Still another embodiment pertains to compositions for treating colon cancer in a mammal with measurably additive antitumorigenesis, said compositions comprising therapeutically effective amounts of bevacizumab and a peptidomimetic of the second of the three Type-1 repeats of TSP-1 or a salt, prodrug, or salt of a prodrug of either or both.

Still another embodiment pertains to separate compositions to be administered together for treating colon cancer in a mammal with measurably additive antitumorigenesis, one comprising a therapeutically effective amount of bevacizumab and the other comprising a peptidomimetic of the second of the three Type-1 repeats of TSP-1 or a salt, prodrug, or salt of a prodrug thereof.

Still another embodiment pertains to compositions for treating colon cancer in a mammal with measurably additive antitumorigenesis, said compositions comprising therapeutically effective amounts of bevacizumab and Sar-Gly-Val-D-alloIle-Thr-Nva-Ile-Arg-Pro, or a salt, prodrug, or salt of a prodrug thereof.

Still another embodiment pertains to separate compositions to be administered together for treating colon cancer in a mammal with measurably additive antitumorigenesis, one comprising a therapeutically effective amount of bevacizumab and the other comprising Sar-Gly-Val-D-alloIle-Thr-Nva-Ile-Arg-Pro or a salt, prodrug, or salt of a prodrug thereof.

Still another embodiment pertains to methods for treating angiogenic diseases in a mammal with measurably additive antitumorigenesis, said methods comprising administering to the mammal therapeutically effective amounts of a VEGF inhibitor and a peptidomimetic of the second of the three Type-1 repeats of TSP-1 or a salt, prodrug or salt of a prodrug of either or both.

Still another embodiment pertains to methods for treating angiogenic diseases in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and a VEGF inhibitor or a salt, prodrug or salt of a prodrug thereof on at least days one, three, eight and ten.

Still another embodiment pertains to methods for treating angiogenic diseases in a mammal with measurably additive antitumorigenesis, said methods comprising administering to the mammal therapeutically effective amount of a VEGF inhibitor and Sar-Gly-Val-D-alloIle-Thr-Nva-Ile-Arg-Pro or a salt, prodrug or salt of a prodrug of either or both.

Still another embodiment pertains to methods for treating angiogenic diseases in a mammal comprising administering to the mammal a therapeutically effective amount of Sar-Gly-Val-D-alloIle-Thr-Nva-Ile-Arg-Pro or a salt, prodrug or salt of a prodrug thereof at least once per day for at least fourteen days and a VEGF inhibitor or a salt, prodrug or salt of a prodrug thereof on at least days one, three, eight and ten.

Still another embodiment pertains to methods for treating angiogenic diseases in a mammal with measurably additive antitumorigenesis, said methods comprising administering to the mammal therapeutically effective amounts of bevacizumab and a peptidomimetic of the second of the three Type-1 repeats of TSP-1 or a salt, prodrug or salt of a prodrug thereof.

Still another embodiment pertains to methods for treating angiogenic diseases in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and bevacizumab on at least days one, three, eight and ten.

Still another embodiment pertains to methods for treating angiogenic diseases in a mammal with measurably additive antitumorigenesis, said methods comprising administering to the mammal therapeutically effective amounts of bevacizumab and Sar-Gly-Val-D-alloIle-Thr-Nva-Ile-Arg-Pro or a salt, prodrug or salt of a prodrug thereof.

Still another embodiment pertains to methods for treating angiogenic diseases in a mammal comprising administering to the mammal a therapeutically effective amount of Sar-Gly-Val-D-alloIle-Thr-Nva-Ile-Arg-Pro or a salt, prodrug or salt of a prodrug thereof at least once per day for at least fourteen days and bevacizumab on at least days one, three, eight and ten.

Still another embodiment pertains to methods for inhibiting tumor growth in a mammal with measurably additive antitumorigenesis, said methods comprising administering to the mammal therapeutically effective amounts of a VEGF inhibitor and a peptidomimetic of the second of the three Type-1 repeats of TSP-1 or a salt, prodrug or salt of a prodrug of either or both.

Still another embodiment pertains to methods for inhibiting tumor growth in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and a VEGF inhibitor or a salt, prodrug or salt of a prodrug thereof on at least days one, three, eight and ten.

Still another embodiment pertains to methods for inhibiting tumor growth in a mammal with measurably additive antitumorigenesis, said methods comprising administering to the mammal therapeutically effective amounts of a VEGF inhibitor and Sar-Gly-Val-D-alloIle-Thr-Nva-Ile-Arg-Pro or a salt, prodrug or salt of a prodrug thereof.

Still another embodiment pertains to methods for inhibiting tumor growth in a mammal comprising administering to the mammal a therapeutically effective amount of Sar-Gly-Val-D-alloIle-Thr-Nva-Ile-Arg-Pro or a salt, prodrug or salt of a prodrug thereof at least once per day for at least fourteen days and a VEGF inhibitor or a salt, prodrug or salt of a prodrug thereof on at least days one, three, eight and ten.

Still another embodiment pertains to methods for inhibiting tumor growth in a mammal with measurably additive antitumorigenesis, said methods comprising administering to the mammal therapeutically effective amounts of bevacizumab and a peptidomimetic of the second of the three Type-1 repeats TSP-1 or a salt, prodrug or salt of a prodrug of either or both.

Still another embodiment pertains to methods for inhibiting tumor growth in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and bevacizumab on at least days one, three, eight and ten.

Still another embodiment pertains to methods for inhibiting tumor growth in a mammal with measurably additive antitumorigenesis, said methods comprising administering to the mammal therapeutically effective amounts of bevacizumab and Sar-Gly-Val-D-alloIle-Thr-Nva-Ile-Arg-Pro or a salt, prodrug or salt of a prodrug thereof.

Still another embodiment pertains to methods for inhibiting tumor growth in a mammal comprising administering to the mammal a therapeutically effective amount of Sar-Gly-Val-D-alloIle-Thr-Nva-Ile-Arg-Pro or a salt, prodrug or salt of a prodrug thereof at least once per day for at least fourteen days and bevacizumab on at least days one, three, eight and ten.

Still another embodiment pertains to methods for treating fibrosarcoma in a mammal with measurably additive antitumorigenesis, said methods comprising administering to the mammal therapeutically effective amounts of a VEGF inhibitor and a peptidomimetic of the second of the three Type-1 repeats of TSP-1 or a salt, prodrug or salt of a prodrug of either or both.

Still another embodiment pertains to methods for treating fibrosarcoma in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and a VEGF inhibitor or a salt, prodrug or salt of a prodrug thereof on at least days one, three, eight and ten.

Still another embodiment pertains to methods for treating fibrosarcoma in a mammal with measurably additive antitumorigenesis, said methods comprising administering to the mammal therapeutically effective amounts of a VEGF inhibitor and Sar-Gly-Val-D-alloIle-Thr-Nva-Ile-Arg-Pro or a salt, prodrug or salt of a prodrug of either or both.

Still another embodiment pertains to methods for treating fibrosarcoma in a mammal comprising administering to the mammal a therapeutically effective amount of Sar-Gly-Val-D-alloIle-Thr-Nva-Ile-Arg-Pro or a salt, prodrug or salt of a prodrug thereof at least once per day for at least fourteen days and a VEGF inhibitor or a salt, prodrug or salt of a prodrug thereof on at least days one, three, eight and ten.

Still another embodiment pertains to methods for treating fibrosarcoma in a mammal with measurably additive antitumorigenesis, said methods comprising administering to the mammal therapeutically effective amounts of bevacizumab and a peptidomimetic of the second of the three Type-1 repeats of TSP-1 or a salt, prodrug or salt of a prodrug thereof.

Still another embodiment pertains to methods for treating fibrosarcoma in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and bevacizumab on at least days one, three, eight and ten.

Still another embodiment pertains to methods for treating fibrosarcoma in a mammal with measurably additive antitumorigenesis, said methods comprising administering to the mammal therapeutically effective amounts of bevacizumab and Sar-Gly-Val-D-alloIle-Thr-Nva-Ile-Arg-Pro or a salt, prodrug or salt of a prodrug thereof.

Still another embodiment pertains to methods for treating fibrosarcoma in a mammal comprising administering to the mammal a therapeutically effective amount of Sar-Gly-Val-D-alloIle-Thr-Nva-Ile-Arg-Pro or a salt, prodrug or salt of a prodrug thereof at least once per day for at least fourteen days and bevacizumab on at least days one, three, eight and ten.

Still another embodiment pertains to methods for treating colon cancer in a mammal with measurably additive antitumorigenesis, said methods comprising administering to the mammal therapeutically effective amounts of a VEGF inhibitor and a peptidomimetic of the second of the three Type-1 repeats of TSP-1 or a salt, prodrug or salt of a prodrug of either or both.

Still another embodiment pertains to methods for treating colon cancer in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and a VEGF inhibitor or a salt, prodrug or salt of a prodrug thereof on at least days one, three, eight and ten.

Still another embodiment pertains to methods for treating colon cancer in a mammal with measurably additive antitumorigenesis, said methods comprising administering to the mammal therapeutically effective amounts of a VEGF inhibitor and Sar-Gly-Val-D-alloIle-Thr-Nva-Ile-Arg-Pro or a salt, prodrug or salt of a prodrug of either or both.

Still another embodiment pertains to methods for treating colon cancer in a mammal comprising administering to the mammal a therapeutically effective amount of Sar-Gly-Val-D-alloIle-Thr-Nva-Ile-Arg-Pro or a salt, prodrug or salt of a prodrug thereof at least once per day for at least fourteen days and a VEGF inhibitor or a salt, prodrug or salt of a prodrug thereof on at least days one, three, eight and ten.

Still another embodiment pertains to methods for treating colon cancer in a mammal with measurably additive antitumorigenesis, said methods comprising administering to the mammal therapeutically effective amounts of bevacizumab and a peptidomimetic of the second of the three Type-1 repeats of TSP-1 or a salt, prodrug or salt of a prodrug thereof.

Still another embodiment pertains to methods for treating colon cancer in a mammal comprising administering to the mammal a therapeutically effective amount of a peptidomimetic of the second of the three Type-1 repeats of TSP-1 or a salt, prodrug or salt of a prodrug thereof at least once per day for at least fourteen days and bevacizumab on at least days one, three, eight and ten.

Still another embodiment pertains to methods for treating colon cancer in a mammal with measurably additive antitumorigenesis, said methods comprising administering to the mammal therapeutically effective amounts of bevacizumab and Sar-Gly-Val-D-alloIle-Thr-Nva-Ile-Arg-Pro or a salt, prodrug or salt of a prodrug thereof.

Still another embodiment pertains to methods for treating colon cancer in a mammal comprising administering to the mammal a therapeutically effective amount of Sar-Gly-Val-D-alloIle-Thr-Nva-Ile-Arg-Pro or a salt, prodrug or salt of a prodrug thereof at least once per day for at least fourteen days and bevacizumab on at least days one, three, eight and ten.

### DETAILED DESCRIPTION OF THE INVENTION

Variable moieties herein are represented by identifiers (capital letters with numerical and/or alphabetical superscripts) and may be specifically embodied.

Proper valences are maintained for all moieties and combinations thereof of the compounds of this invention.

The term “treating,” as used herein, means at least sustaining and preferably reversing the course of a disease or adverse physiological event.

The term “angiogenesis,” as used herein, means formation of new blood vessels.

The term “cancer,” as used herein, means growth of tumor cells which interfere with the growth of healthy cells. Cancers include, but are not limited to, fibrosarcoma and gastrointestinal cancer such as gastric cancer, colon cancer and the like.

The term “mammal,” as used herein, means a particular class of vertebrate.

The term “measurably additive antiangiogenic effect,” as used herein means greater antitumorigenesis than obtained from use of either a VEGF inhibitor or a peptidomimetic of the second of the three Type-1 repeats of TSP-1 or a salt, prodrug or salt of a prodrug of either or both.

The term “thrombospondin-1,” as used herein, means an antiangiogenic protein which functions by inhibiting endothelial cell proliferation, thereby inducing apoptosis (programmed cell death).

The term “antitumorigenesis,” as used herein, means inhibition of tumor growth.

The term “vascular endothelial growth factor,” as used herein, means an activator of endothelial cell migration and proliferation and a modulator of physiologic and pathologic angiogenesis. Inhibitors of VEGF include, but are not limited to VEGF-Trap, anti-VGF monoclonal antibodies such as bevacizumab (AVASTIN®) and the like.

The term “peptidomimetic of the second of the three Type-1 repeats of TSP-1,” as used herein, means parent peptide Gly-Val-Ile-Thr-Arg-Ile-Arg, the N-terminus Gly of which is capped with R¹-Sar, the He of which is replaced with D-Ile or D-alloIle, the Arg of which is replaced with Nva or Gln and the terminal Arg of which is replaced with Pro-R², wherein R¹ is hydrogen or an N-terminus prodrug-forming moiety, and R² is hydrogen or a C-terminus prodrug-forming moiety.

Compounds of this invention contain amino acids having asymmetrically substituted carbon atoms in the L- or D-configuration, wherein amino acids having the L-configuration are those which occur naturally. Atoms with an excess of one configuration over the other are assigned the configuration which is present in the higher amount, preferably an excess of about 85%-90%, more preferably an excess of about 95%-99%, and still more preferably an excess greater than about 99.5%.

The term “D-alloIle,” as used herein, means D-alloisolucyl.

The term “Arg,” as used herein, means L-argininyl.

The term “Gly,” as used herein, means L-glycyl.

The term “Gln,” as used herein, means L-glutamine.

The term “Ile,” as used herein, means L-isolucyl.

The term “Nva,” as used herein, means L-norvalinyl.

The term “Pro,” as used herein, means L-prolinyl.

The term “Sar,” as used herein, means L-sarcosyl (N-methyl-L-glycyl).

The term “Thr,” as used herein, means L-threoninyl.

The term “Val,” as used herein, means L-valyl.

The term “drugs of this invention,” as used herein, means VEGF inhibitors and peptidomimetics of the second of the three Type-1 repeats of TSP-1.

The term “prodrugs of this invention,” as used herein, means VEGF inhibitors and peptidomimetics of the second of the three Type-1 repeats of TSP-1 having attached thereto at least one prodrug-forming moiety.

Drugs of this invention may exist as an acid addition salts, basic addition salts or zwitterions. Acid addition salts are those derived from the reaction of the compounds with an acid. For example, the acetate, adipate, alginate, bicarbonate, citrate, aspartate, benzoate, benzenesulfonate, bisulfate, butyrate, camphorate, camphorsufonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetic, trifluoroacetic, para-toluenesulfonate, and undecanoate salts of the drugs of this invention, and prodrugs thereof, are contemplated as being embraced by this invention. Basic addition salts of the drugs of this invention are those derived from the reaction of the same with the hydroxide, carbonate or bicarbonate of cations such as lithium, sodium, potassium, calcium and magnesium.

Drugs of this invention may be administered, for example, parenterally (intramuscularly, intraperintoneally intrasternally, intravenously subcutaneously) or transdermally.

Therapeutically effective amounts of drugs of this invention depend on the recipient of treatment, the cancer being treated and severity thereof, compositions containing them, time of administration, route of administration, duration of treatment, their potency, their rate of clearance and whether or not other drugs are co-administered. The amount of a compound of a drug of this invention used to make a composition to be administered daily to a patient in a single dose or in divided doses is from about 0.05 to about 300 mg/kg (mpk) body weight. Single dose compositions contain these amounts or a combination of submultiples thereof.

Drugs of this invention may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising drugs of this invention to be administered parenterally or transdermally include, for example, 1,3-butanediol, castor oil, corn oil, cottonseed oil, dextrose, 5% glucose in water (D5W), germ oil, groundnut oil, isotonic sodium chloride solution (0.9% sodium chloride in water), liposomes, oleic acid, olive oil, peanut oil, Ringer's solution, safflower oil, sesame oil, soybean oil, U.S.P. or, water, mixtures thereof and the like.

Drugs of this invention containing NH, C(O)OH, OH or SH moieties may have attached thereto prodrug-forming moieties which are removed by metabolic processes and release the compounds having the freed NH, C(O)OH, OH or SH in vivo. Prodrugs of this invention may have modified or improved properties such as solubility and/or hydrophobicity, absorption in the gastrointestinal tract, bioavailability, reduction of site-of-administration irritation, tissue penetration, rate of clearance and the like.

In a preferred embodiment for the practice of this invention, the N-terminus (sarcosinyl) and the C-terminus (prolyl) of the representative peptidomimetic of the second of the three Type-1 repeats of TSP-of this invention have attached thereto an acetyl (CH₃C(O) or Ac) and an ethylamino moiety, respectively.

Other N-terminus prodrug forming groups include, but are not limited to, acetoxy (CH₃CO(O)), benzoyl (C₆H₅C(O)), benzoyloxy (C₆H₅CO(O)) and the like. Other C-terminus prodrug forming groups include, but are not limited to, ethyl, diethylamino and the like.

The following examples are presented to provide what is believed to be the most useful and readily understood description of procedures and conceptual aspects of this invention.

Female SCID mice (Charles River Laboratories, Wilmington, Mass., USA) with an average body weight of 18.7 g were inoculated subcutaneously into the right flank with 0.1 mL of about 0.5×10⁶ HT-1080 cells (1:1 matrigel) on a Friday. Day 3, post inoculation (Monday), the mice were ear tagged and randomized into groups of ten control and treated groups. The representative prodrug of a peptidomimetic of the second of the three Type-1 repeats of TSP-1, N-Ac-Sar-Gly-Val-D-alloIle-Thr-Nva-Ile-Arg-Pro-NHCH₂CH₃ (120 mpk), was administered twice per day (BID) during the dosing regimen, and the representative VGF inhibitor bevacizumab (3 mpk) was administrated each Monday and Wednesday during the same dosing regimen. Tumors were measured with calipers every Monday, Wednesday and Friday days beginning at day 3, post inoculation. Tumor volumes were calculated according to the formula V=L×W²/2, wherein V is volume (mm³), L is length and W is width. Antitumorigenesis was measured by percent tumor growth inhibition (TGI). Results of the study are shown in TABLES 1A-D.

The term “mpk,” as used herein, means milligrams drug per kilogram mammal.

The term “SE,” as used herein, means standard error.

The term “T/C,” as used herein, means size of tumor (treated/control).

The term “SC,” as used herein, means subcutaneously.

The term “p-value,” as used herein, means confidence level of comparison to control. For example, a p-value less than 0.5 means having greater than 95% confidence that the result did not occur randomly.

Controls comprised vehicle only. Vehicles for N-Ac-Sar-Gly-Val-D-alloIle-Thr-Nva-Ile-Arg-Pro-NHCH₂CH₃ and bevacizumab were D5W (5% glucose in water) and isotonic saline (0.9% NaCl in water), respectively.

TABLE 1A
Control (0 Mpk) BID + Twice Per Week

| | Mean Tumor Volume | % T/C (% TGI) |
|---|---|---|
| | mm³ ± SE | Days 7-14 |
| Day 7 | 0.41 ± 0.05 | 100 |
| Day 10 | 0.90 ± 0.09 | 100 |
| Day 12 | 1.27 ± 0.13 | 100 |
| Day 14 | 2.02 ± 0.18 | 100 |

TABLE 1B
N—Ac-Sar-Gly-Val-D-alloIle-Thr-Nva-Ile-Arg-Pro-NHCH₂CH₃
120 Mpk (BID, SC)

| | Mean Tumor Volume | % T/C (% TGI) | Student's t-test |
|---|---|---|---|
| | mm³ ± SE | Days 7-14 | p-value |
| Day 7 | 0.28 ± 0.02 | 69.76 (30.24) | 0.01483 |
| Day 10 | 0.53 ± 0.05 | 58.39 (41.61) | 0.00093 |
| Day 12 | 0.71 ± 0.07 | 55.70 (44.30) | 0.00060 |
| Day 14 | 1.03 ± 0.10 | 51.21 (48.79) | 0.00007 |

TABLE 1C
Bevacizumab
3.0 Mpk (Twice Per Week, SC)

| | Mean Tumor Volume | % T/C (% TGI) | Student's t-test |
|---|---|---|---|
| | mm³ ± SE | Days 7-14 | p-value |
| Day 7 | 0.27 ± 0.02 | 66.32 (33.68) | 0.00901 |
| Day 10 | 0.55 ± 0.07 | 60.60 (39.40) | 0.00297 |
| Day 12 | 0.77 ± 0.06 | 60.75 (39.25) | 0.00117 |
| Day 14 | 0.84 ± 0.05 | 41.70 (58.30) | 0.00000 |
| Day 17 | 1.17 ± 0.11 | | |
| Day 19 | 1.35 ± 0.11 | | |

TABLE 1D
N—Ac-Sar-Gly-Val-D-alloIle-Thr-Nva-Ile-Arg-Pro-NHCH₂CH₃
120 Mpk (BID, SC) + Bevacizumab
3.0 Mpk (Twice Per Week, SC)

| | Mean Tumor Volume | % T/C (% TGI) | Student's t-test |
|---|---|---|---|
| | mm³ ± SE | Days 7-14 | p-value |
| Day 7 | 0.18 ± 0.03 | 44.16 (55.84) | 0.00028 |
| Day 10 | 0.39 ± 0.03 | 42.98 (57.02) | 0.00003 |
| Day 12 | 0.50 ± 0.07 | 39.23 (60.77) | 0.00002 |
| Day 14 | 0.55 ± 0.04 | 27.28 (72.72) | 0.00000 |
| Day 17 | 0.84 ± 0.05 | | |
| Day 19 | 1.28 ± 0.15 | | |

TABLE 1E
Comparison of single drug treatment and combination drug treatment
N—Ac-Sar-Gly-Val-D-alloIle-Thr-Nva-Ile-Arg-Pro-NHCH₂CH₃ = peptidomimetic
and Bevacizumab

| | | | Treatment Difference | |
|---|---|---|---|---|
| Time in | Single Drug | Combination | (Treatment 2 − Treament 1) | |
| days | Treatment 1 | Treatment 2 | in mm³ | p-value # |
| 7 | Peptidomimetic, | Peptidomimetic | 83 | 0.0109 |
| | 120 mg/kg | (120 mg/kg) + Avastin | | |
| | | (3 mg/kg) | | |
| 7 | Avastin, | Peptidomimetic | 69 | 0.0294 |
| | 3 mg/kg | (120 mg/kg) + Avastin | | |
| | | (3 mg/kg) | | |
| 10 | Peptidomimetic, | Peptidomimetic | 132 | 0.0452 |
| | 120 mg/kg | (120 mg/kg) + Avastin | | |
| | | (3 mg/kg) | | |
| 10 | Avastin, | Peptidomimetic | 130 | 0.0486 |
| | 3 mg/kg | (120 mg/kg) + Avastin | | |
| | | (3 mg/kg) | | |
| 12 | Peptidomimetic, | Peptidomimetic | 212 | 0.0265 |
| | 120 mg/kg | (120 mg/kg) + Avastin | | |
| | | (3 mg/kg) | | |
| 12 | Avastin, | Peptidomimetic | 279 | 0.0061 |
| | 3 mg/kg | (120 mg/kg) + Avastin | | |
| | | (3 mg/kg) | | |
| 14 | Peptidomimetic, | Peptidomimetic | 451 | <.0001 |
| | 120 mg/kg | (120 mg/kg) + Avastin | | |
| | | (3 mg/kg) | | |
| 14 | Avastin, | Peptidomimetic | 287 | 0.0007 |
| | 3 mg/kg | (120 mg/kg) + Avastin | | |
| | | (3 mg/kg) | | |
| 17 | Avastin, | Peptidomimetic | 290 | 0.0097 |
| | 3 mg/kg | (120 mg/kg) + Avastin | | |
| | | (3 mg/kg) | | |
| 19 | Avastin, | Peptidomimetic | 78 | 0.6394 |
| | 3 mg/kg | (120 mg/kg) + Avastin | | |
| | | (3 mg/kg) | | |

The data from TABLES 1A-E show greater antitumorigenesis obtained with bevacizumab and the prodrug of Sar-Gly-Val-D-alloIle-Thr-Nva-Ile-Arg-Pro than from use of either alone, i.e. an additive antitumorigenesis.

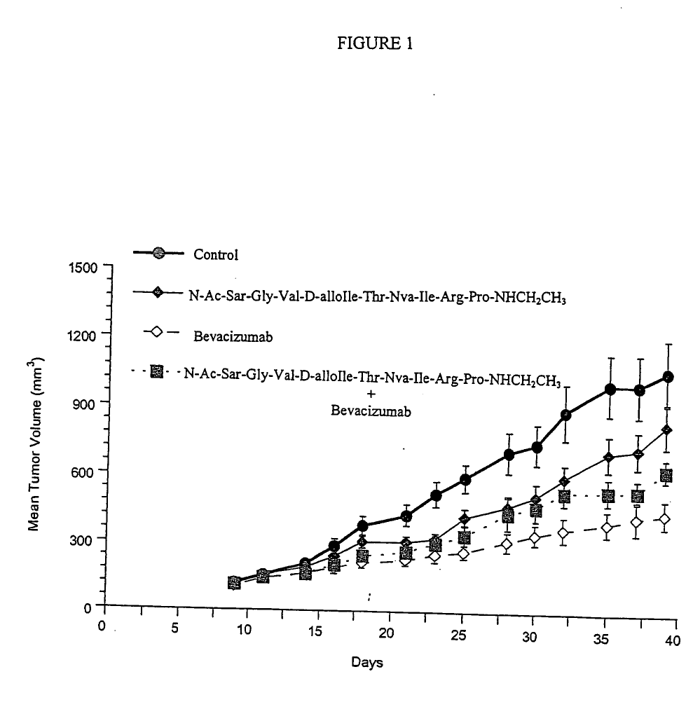

Female SCID mice (Charles River Laboratories, Wilmington, Mass., USA) with an average body weight of 18.7 g were inoculated subcutaneously into the right flank with 0.1 mL of about 0.5×10⁶ HT29 cells (1:1 matrigel) on a Friday. Day 3, post inoculation (Monday), the mice were ear tagged and randomized into groups of ten control and treated groups. The representative prodrug of a peptidomimetic of the second of the three Type-1 repeats of TSP-1, N-Ac-Sar-Gly-Val-D-alloIle-Thr-Nva-Ile-Arg-Pro-NHCH₂CH₃ (240 mpk), was administered twice per day (BID) during the dosing regimen, and the representative VGF inhibitor bevacizumab (3 mpk) was administrated each Monday and Wednesday during the same dosing regimen. Tumors were measured with calipers every Monday, Wednesday and Friday days beginning at day 3, post inoculation. Tumor volumes were calculated according to the formula V=L×W²/2, wherein V is volume (mm³), L is length and W is width. Results of the study, shown in FIG. 1, show greater antitumorigenesis obtained with bevacizumab and the prodrug of Sar-Gly-Val-D-alloIle-Thr-Nva-Ile-Arg-Pro than from use of either alone, i.e. an additive antitumorigenesis.

The foregoing is meant to illustrate the invention and not limit it to the disclosed embodiments. Variations and changes obvious to one skilled in the art are intended to be within the scope and nature of the invention as defined in the claims.

We claim:

1. A composition for treating cancer in a mammal said composition comprising therapeutically effective amounts of a vascular endothelial growth factor (VEGF) inhibitor and a peptidomimetic of the second of the three Type-1 repeats of thrombospondin-1 (TSP-1) or a salt, prodrug or salt of a prodrug of either or both.

2. A composition for treating cancer in a mammal said composition comprising therapeutically effective amounts of a VEGF inhibitor and Sar-Gly-Val-D-alloIle-Thr-Nva-Ile-Arg-Pro or a salt, prodrug or salt of a prodrug of either or both.

3. A composition for treating cancer in a mammal said composition comprising therapeutically effective amounts of bevacizumab and a peptidomimetic of the second of the three Type-1 repeats of TSP-1 or a salt, prodrug, or salt of a prodrug thereof.

4. A method for inhibiting tumor growth in a mammal said method comprising administering to the mammal therapeutically effective amounts of bevacizumab and Sar-Gly-Val-D-alloIle-Thr-Nva-Ile-Arg-Pro or a salt, prodrug or salt of a prodrug thereof.

5. A method for treating fibrosarcoma in a mammal, said method comprising administering to the mammal therapeutically effective amounts of bevacizumab and Sar-Gly-Val-D-alloIle-Thr-Nva-Ile-Arg-Pro or a salt, prodrug or salt of a prodrug thereof.

6. A method for treating treating colon cancer in a mammal, said method comprising administering to the mammal therapeutically effective amounts of bevacizumab and Sar-Gly-Val-D-alloIle-Thr-Nva-Ile-Arg-Pro or a salt, prodrug or salt of a prodrug thereof.
